(19) United States
(12) Patent Application Publication
(10) Pub. No.: US 2018/0128886 A1
(43) Pub. Date: May 10, 2018

(54) MAGNETIC SENSOR AND CELL UNIT

(71) Applicant: SEIKO EPSON CORPORATION, Tokyo (JP)

(72) Inventor: Kimio NAGASAKA, Hokotu-shi (JP)

(73) Assignee: SEIKO EPSON CORPORATION, Tokyo (JP)

(21) Appl. No.: 15/796,004

(22) Filed: Oct. 27, 2017

(30) Foreign Application Priority Data
Nov. 4, 2016 (JP) 2016-216089

(51) Int. Cl. G01R 33/032; A61B 5/04

(52) U.S. Cl. CPC G01R 33/032; A61B 2562/0223; A61B 5/04008; A61B 5/04007

(57) ABSTRACT

A magnetic sensor includes a light flux emitting unit, a first cell onto which a light flux, which propagates in a first direction, is incident and that accommodates a medium which changes optical characteristics of the light flux depending on a magnitude of a magnetic field, a first light flux bender that bends some of the plurality of light fluxes in a second direction different from the first direction, a second cell onto which the light flux, which is bent in the second direction in the first light flux bender, is incident and that accommodates a medium which changes optical characteristics of the light flux depending on the magnitude of the magnetic field, a first light detection element that detects optical characteristics of the light flux emitted from the first cell, and a second light detection element that detects optical characteristics of the light flux emitted from the second cell.

### BACKGROUND

### 1. Technical Field

The present invention relates to a magnetic sensor and a cell unit.

### 2. Related Art

A magnetic field measurement apparatus for measuring biomagnetic fields of a living body such as a magnetic field of a heart (heart magnetic field) or a magnetic field of a brain (brain magnetic field) weaker than geomagnetism is known. The magnetic field measurement apparatus is a non-invasive measurement apparatus and thus, it is possible to confirm a condition of an internal organ by the magnetic field measurement apparatus without putting loads on a subject (living body). In such a magnetic field measurement apparatus, a magnetic sensor capable of detecting components in three axial directions of the magnetic field is required in order to measure the magnetic field distributed in three-dimensional space.

For example, in a literature of S.J. Selter and M.V. Romalis, “Unshielded three-axis vector operation of a spin-exchange-relaxation-free atomic magnetometer”, APPLIED PHYSICS LETTERS, VOLUME 85, NUMBER 20, p. 4804-4806, 15 NOV. 2004, AC magnetic field β_{X}^{mod}sin(ω_{X}t) is applied to a cell whose detection axis corresponds to the Y-axis direction in the X-axis direction and lock-in detection is performed so that a Z-axis direction component β_{Z}⁰ can be measured and the AC magnetic field β_{X}^{mod}sin(ω_{Z}t) is applied to the cell in the Z-axis direction and lock-in detection is performed so that an X-axis direction component β_{X}⁰ can be measured. In such a magnetic sensor, it is possible to detect components in the three axial directions of a magnetic field.

However, in the literature, three pairs of Helmholtz coils for detecting the X-axis direction component, the Y-axis direction component, and the Z-axis direction component of the magnetic field are provided and a configuration of the magnetic sensor may be complicated. Furthermore, in the literature, an electromagnetic interference phenomenon may occur between a plurality of Helmholtz coils and the magnetic field may not be accurately detected.

### SUMMARY

An advantage of some aspects of the invention is to provide a magnetic sensor capable of detecting components in a plurality of directions of a magnetic field and accurately detecting the magnetic field by a simple configuration. Another advantage of some aspects of the invention is to provide a cell unit capable of detecting components in a plurality of directions of a magnetic field and accurately detecting the magnetic field by a simple configuration.

A magnetic sensor according to an aspect of the invention includes a light flux emitting unit that emits a plurality of light fluxes, a first cell onto which a light flux, which is emitted from the light flux emitting unit and which propagates in a first direction, is incident and that accommodates a medium which changes optical characteristics of the light flux depending on a magnitude of a magnetic field, a first light flux bender that bends some of the plurality of light fluxes emitted from the light flux emitting unit in a second direction different from the first direction, a second cell onto which the light flux, which is bent in the second direction in the first light flux bender, is incident and that accommodates a medium which changes optical characteristics of the light flux depending on the magnitude of the magnetic field, a first light detection element that detects optical characteristics of a light flux emitted from the first cell, and a second light detection element that detects optical characteristics of a light flux emitted from the second cell.

In the magnetic sensor, for example, even when a plurality of Helmholtz coil pairs are not provided in order to detect components in a plurality of directions of the magnetic field, components in a plurality of directions of the magnetic field may be detected. Accordingly, in the magnetic sensor, components in a plurality of directions of the magnetic field may be detected and the magnetic field may be accurately detected with a simple configuration.

The magnetic sensor according to the aspect of the invention may further include a second light flux bender that bends some of the plurality of light fluxes emitted from the light flux emitting unit in a third direction different from the first direction and the second direction, a third cell onto which the light flux, which is bent in the third direction in the second light flux bender, is incident and that accommodates a medium which changes optical characteristics of the light flux depending on the magnitude of the magnetic field, and a third light detection element that detects optical characteristics of the light flux emitted from the third cell.

In the magnetic sensor with this configuration, components in three directions of the magnetic field may be detected.

In the magnetic sensor according to the aspect of the invention, the first direction, the second direction, and the third direction may be orthogonal to each other.

In the magnetic sensor with this configuration, components of the magnetic field in three axial directions orthogonal to each other may be detected.

In the magnetic sensor according to the aspect of the invention, any of the first cell, the second cell, and the third cell is provided with a quantity of three or more, and all of the centers of the cells provided with a quantity of three or more may not be aligned on a straight line.

In the magnetic sensor with this configuration, components of the magnetic field in three axial directions orthogonal to each other may be more surely detected.

In the magnetic sensor according to the aspect of the invention, the number of the second cells and the number of the third cells may be the same.

In the magnetic sensor with this configuration, a component in the second axial direction and a component in the third axial direction of the magnetic field may be detected with the same accuracy.

In the magnetic sensor according to the aspect of the invention, the first cells, the second cells, and the third cells may be provided on the same plane.

In the magnetic sensor with this configuration, the first cell, the second cell, and the third cell may be easily supported by, for example, a single substrate.

The magnetic sensor according to the aspect of the invention may further include a light flux guide portion that guides the light flux emitted from the second cell to the second light detection element.

In the magnetic sensor with this configuration, the light flux emitted from the second cell may be made incident on the first light flux guide portion by the first light flux guide portion.

In the magnetic sensor according to the aspect of the invention, the light flux guide portion is a phase compensation mirror reflecting the light flux emitted from the second cell and the light flux guide portion may reflect the light flux while maintaining a phase difference between P wave and S wave of the light flux, of which a polarization plane is rotated, as it is.

In the magnetic sensor with this configuration, decrease in sensitivity of the second light detection element may be suppressed.

In the magnetic sensor according to the aspect of the invention, the light flux emitting unit may emit the plurality of light fluxes in the first direction.

In the magnetic sensor with this configuration, the light flux propagating in the first direction may be made incident on the first cell without using the light flux bender.

In the magnetic sensor according to the aspect of the invention, the medium may be gaseous alkali metal.

In the magnetic sensor with this configuration, alkali metals interact with an applied magnetic field such that a polarization plane of light transmitted through the first cell, the second cell, or the third cell may be changed depending on the magnitude of the magnetic field.

A cell unit according to an aspect of the invention includes a first cell that accommodates a medium changing optical characteristics of a light flux depending on a magnitude of a magnetic field, a first light detection element that is provided in a first direction of the first cell and detects optical characteristics of the light flux, a second cell that accommodates a medium changing optical characteristics of the light flux depending on the magnitude of the magnetic field and has a first surface and a second surface opposing to each other in a second direction orthogonal to the first direction, a first reflection mirror that is provided in the first surface side and inclined at 45 degrees to the first surface, a second reflection mirror that is provided in the second surface side and inclined at 45 degrees to the second surface, and a second light detection element that is provided in a first direction of the second reflection mirror and detects optical characteristics of the light flux.

In the cell unit, components in a plurality of directions of the magnetic field may be detected and the magnetic field may be accurately detected with a simple configuration.

### BRIEF DESCRIPTION OF THE DRAWINGS

The invention will be described with reference to the accompanying drawings, wherein like numbers reference like elements.

FIG. 1 is a side view schematically illustrating a magnetic field measurement apparatus according to the present embodiment.

FIG. 2 is a side view schematically illustrating a first magnetic sensor of the magnetic field measurement apparatus according to the present embodiment.

FIG. 3 is a plan view schematically illustrating the first magnetic sensor of the magnetic field measurement apparatus according to the present embodiment.

FIG. 4 is a diagram illustrating an example of a configuration of a processing device of the magnetic field measurement apparatus according to the present embodiment.

FIG. 5 is a plan view schematically illustrating a magnetic sensor according to the present embodiment.

FIG. 6 is a cross-sectional view schematically illustrating the magnetic sensor according to the present embodiment.

FIG. 7 is another cross-sectional view schematically illustrating the magnetic sensor according to the present embodiment.

FIG. 8 is another cross-sectional view schematically illustrating the magnetic sensor according to the present embodiment.

FIG. 9 is another cross-sectional view schematically illustrating the magnetic sensor according to the present embodiment.

FIG. 10 is another plan view schematically illustrating the magnetic sensor according to the present embodiment.

FIG. 11 is a plan view schematically illustrating a magnetic sensor according to a modification example of the present embodiment.

FIG. 12 is a cross-sectional view schematically illustrating the magnetic sensor according to the modification example of the present embodiment.

FIG. 13 is another cross-sectional view schematically illustrating the magnetic sensor according to the modification example of the present embodiment.

FIG. 14 is another cross-sectional view schematically illustrating the magnetic sensor according to the modification example of the present embodiment.

### DESCRIPTION OF EXEMPLARY EMBODIMENTS

In the following, preferred embodiments of the invention will be described in detail using the drawings. The embodiments to be described in the following do not improperly limit contents of the invention described in the appended claims. All of configurations to be described in the following are not always essential requirements of the invention.

### 1. Magnetic Field Measurement Apparatus

### 1. 1. Configuration

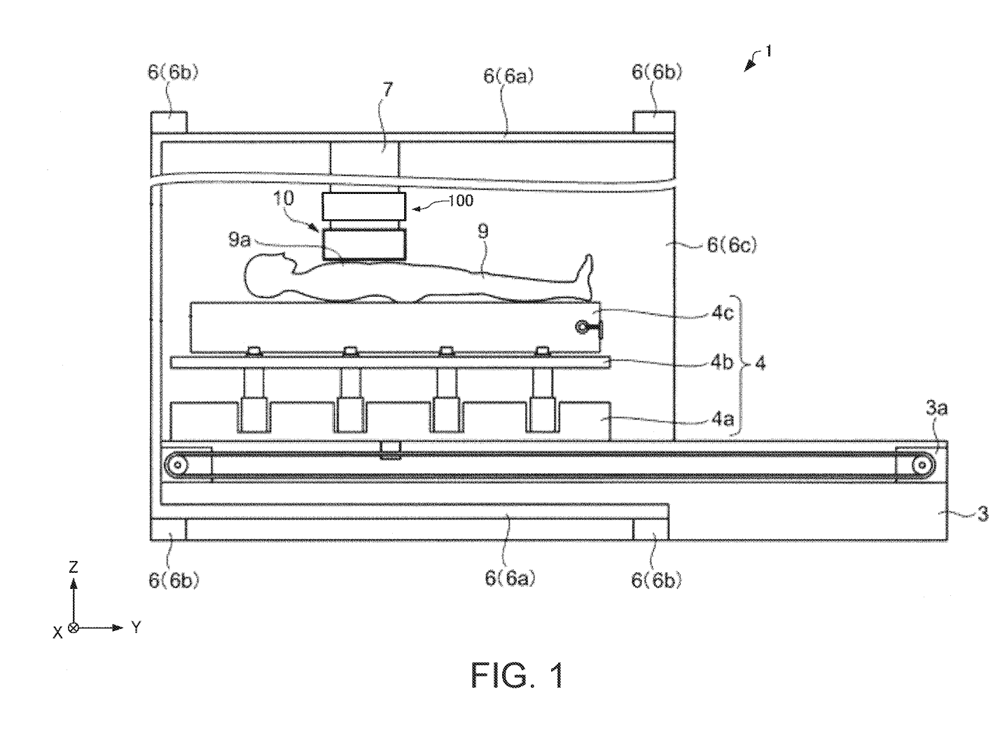

First, a magnetic field measurement apparatus according to the present embodiment will be described with reference to the accompanying drawings. FIG. 1 is a side view schematically illustrating a magnetic field measurement apparatus 1 according to the present embodiment. In FIG. 1 and FIGS. 2 and 3 to be described in the following, the X-axis, the Y-axis, and the Z-axis orthogonal to each other are illustrated as three axes.

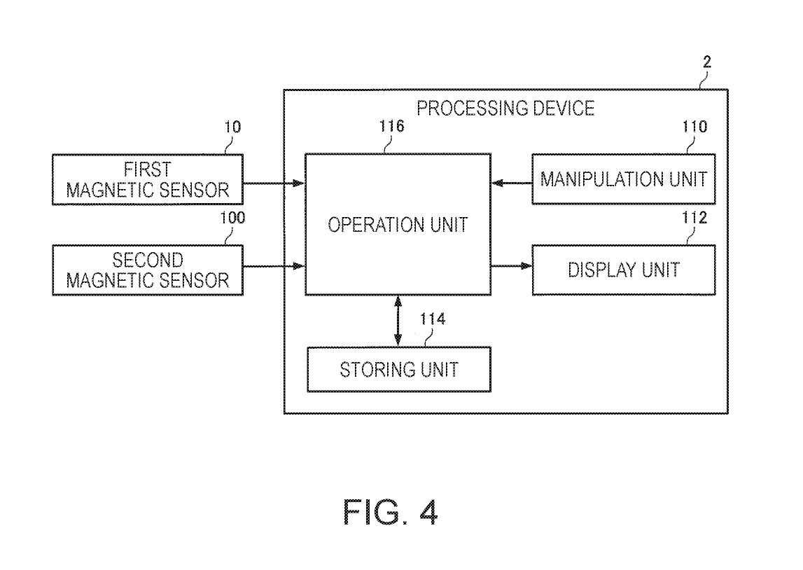

As illustrated in FIG. 1, the magnetic field measurement apparatus 1 is an apparatus that measures a heart magnetic field generated from a subject (living body) 9 as a measurement target, a brain magnetic field generated from the subject (living body) 9, or the like. The magnetic field measurement apparatus 1 includes a first magnetic sensor 10, a magnetic sensor (second magnetic sensor 100 in the illustrated example) according to the invention, a processing device 2 (see FIG. 4), abase 3, a table 4, and a magnetic shield device 6.

The first magnetic sensor 10 is a sensor for measuring a weak magnetic field (magnetic field of the measurement target) such as the heart magnetic field or the brain magnetic field which becomes a measurement target, and is used as a magneto-cardiograph, a magneto-encephalograph, or the like. The second magnetic sensor 100 is a sensor for measuring an environmental magnetic field such as an external magnetic field (magnetic noise). As the first magnetic sensor 10 and the second magnetic sensor 100, for example, an optically pumped type magnetic sensor, a superconducting quantum interference device) (SQUID) magnetic sensor, a flux gate magnetic sensor, an MI sensor, a hole element, and the like are included.

The height direction (up and down direction in FIG. 1) of the magnetic field measurement apparatus 1 is assumed as the Z-axis direction. The Z-axis direction is a vertical direction. The directions in which upper surfaces of the base 3 and the table 4 are extended are assumed as the X-axis direction and the Y-axis direction. The X-axis direction and the Y-axis direction are the horizontal directions and the X-axis direction and the Y-axis direction are directions orthogonal to each other. The height direction (left and right direction in FIG. 1) of the subject 9 which is in a lying state is assumed as the Y-axis direction.

The base 3 is disposed on the bottom surface inside the magnetic shield device 6 (main body 6a) and is extended to the outside of the main body 6a along the Y-axis direction (movable direction of subject 9). The table 4 includes a Y-axis direction table 4a, a Z-axis direction table 4b, and an X-axis direction table 4c. On the base 3, the Y-axis direction table 4a moved along the Y-axis direction by a Y-axis direction linear motion mechanism 3a is installed. On the Y-axis direction table 4a, the Z-axis direction table 4b moved up and down along the Z-axis direction by an elevation device (not illustrated) is installed. On the Z-axis direction table 4b, the X-axis direction table 4c moved on a rail along the X-axis direction by an X-axis direction linear motion mechanism (not illustrated) is installed.

The magnetic shield device 6 is provided with a rectangular main body 6a including an opening portion 6c. The inside of the main body 6a is formed as a cavity and a cross-sectional shape of a side passing through the X-axis direction and the Z-axis direction (plane orthogonal to the Y-axis direction in the X-Z cross section) is a substantially quadrangle. When the heart magnetic field is measured, the subject 9 is accommodated in the main body 6a to be placed on the table 4 in the lying state. The main body 6a is extended in the Y-axis direction and functions as a passive magnetic shield itself.

The first magnetic sensor 10 and the second magnetic sensor 100 are disposed inside the main body 6a of the magnetic shield device 6. The magnetic shield device 6 suppresses a situation that an external magnetic field such as geomagnetism flows into space in which the first magnetic sensor 10 and the second magnetic sensor 100 are disposed. That is, the space in which the first magnetic sensor 10 and the second magnetic sensor 100 are disposed becomes a magnetic field of which the strength thereof is considerably lower than that of the external magnetic field by the magnetic shield device 6 and the influence of the external magnetic field on the first magnetic sensor 10 is suppressed.

The base 3 is protruded from the opening portion 6c of the main body 6a in the +Y direction. The magnetic shield device 6 has a size, for example, a length in the Y-axis direction is approximately 200 cm and one side of the opening portion 6c is approximately 90 cm. The subject 9 lying in the table 4 and the table 4 can be moved on the base 3 along the Y-axis direction to be put in to and out from inside the magnetic shield device 6 through the opening portion 6c.

The processing device 2 (see FIG. 4) is a device that receives an electrical signal from the first magnetic sensor 10 and an electrical signal from the second magnetic sensor 100 and measures the magnetic field such as the heart magnetic field or the brain magnetic field. When a magnetic field or a residual magnetic field is generated due to the electrical signal generated by the processing device 2 and is detected by the first magnetic sensor 10, the detected magnetic field becomes magnetic field noise. For that reason, the processing device 2 is preferably installed on a place separated from the opening portion 6c of the magnetic shield device 6 so that it becomes difficult for the generated magnetic field or the remaining magnetic field to reach the first magnetic sensor 10.

The main body 6a of the magnetic shield device 6 may be formed of a ferromagnetic material having relative permeability of, for example, several thousands or more or a conductor having high conductivity. In the ferromagnetic material, for example, permalloy, ferrite, iron, chromium or cobalt-based amorphous alloy, or the like maybe used. In the conductor having high conductivity, for example, a material having effect of magnetic field reduction by eddy current effect in aluminum or the like can be used. The main body 6a also can be formed by alternately stacking the ferromagnetic material and the conductor having high conductivity.

Correction coils (Helmholtz coil) 6b are installed on ends of the main body 6a in the +Y direction side and the −Y direction side in the base 3. The correction coils 6b have a frame-shape and are disposed to surround the main body 6a. The correction coils 6b are coils for correcting a flow-in magnetic field flowing into internal space of the main body 6a. The flow-in magnetic field indicates a magnetic field passed through the opening portion 6c and flown into the internal space. The magnetic field is strongest in the Y-axis direction with respect to the opening portion 6c. The correction coils 6b generate a magnetic field by currents supplied from the processing device 2 so as to cancel the flow-in magnetic field.

The first magnetic sensor 10 is fixed to a ceiling of the main body 6a through a support member 7. In the illustrated example, the first magnetic sensor 10 is positioned closer to the subject 9 side than the second magnetic sensor 100. The first magnetic sensor 10 detects the component in the Z-axis direction of the magnetic field. That is, the detection axis of the first magnetic sensor 10 is directed to the Z-axis direction. When the heart magnetic field of the subject 9 is measured, the Y-axis direction table 4a and the X-axis direction table 4c are moved so that a chest portion 9a which is a measurement position in the subject 9 is positioned to oppose the first magnetic sensor 10 to move up the Z-axis direction table 4b so as to allow the chest portion 9a to come close to the first magnetic sensor 10.

The second magnetic sensor 100 is fixed to the ceiling of the main body 6a through the support member 7. In the illustrated example, the second magnetic sensor 100 is separated from the first magnetic sensor 10 and is positioned further away from the subject 9 than the first magnetic sensor 10. The second magnetic sensor 100 detects components in the X-axis direction, the Y-axis direction, and the Z-axis direction of the magnetic field. That is, the detection axes of the second magnetic sensor 100 are directed to the X-axis direction, the Y-axis direction, and the Z-axis direction.

### 1. 2. Configuration of First Magnetic Sensor

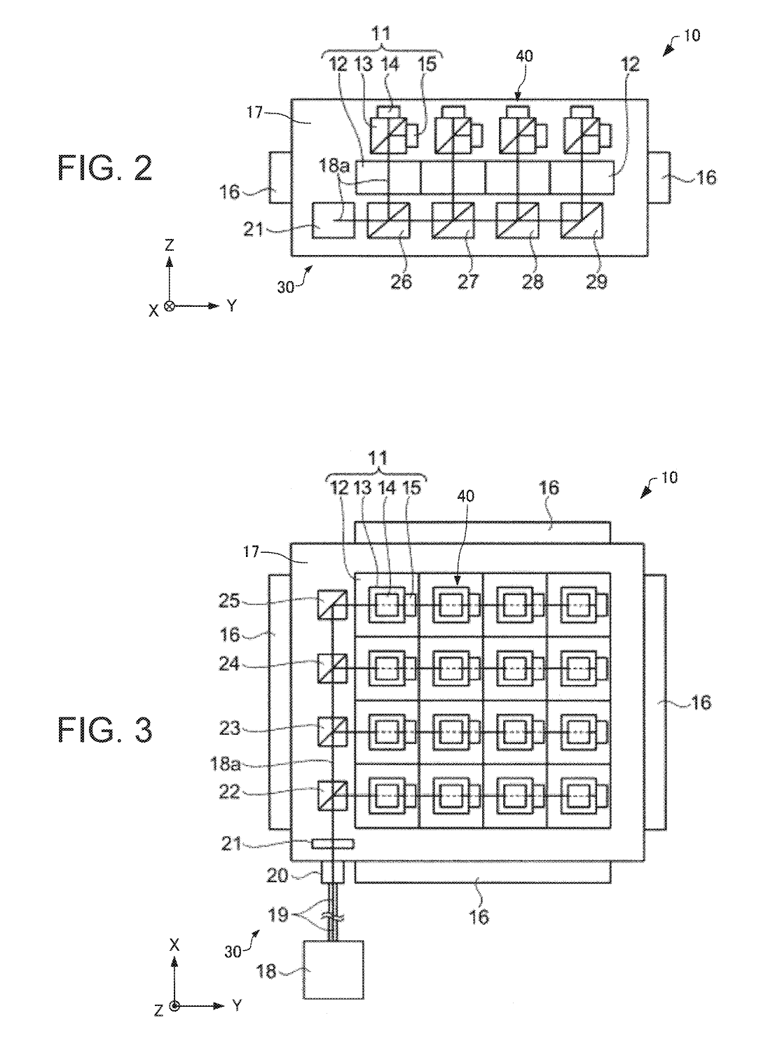

FIG. 2 is a side view schematically illustrating the first magnetic sensor 10. FIG. 3 is a plan view schematically illustrating the first magnetic sensor 10.

As illustrated in FIG. 3, the first magnetic sensor 10 includes a laser light source 18. Laser light 18a emitted from the laser light source 18 is supplied to a substrate (transparent substrate) 17 through an optical fiber 19. The substrate 17 and the optical fiber 19 are connected to each other through an optical connector 20.

The laser light source 18 outputs (emits), for example, laser light 18a having a wavelength according to absorption lines of cesium (Cs). The wavelength of laser light 18a is not particularly limited, but in the present embodiment, is set to a wavelength of, for example, 894 nm corresponding to the D1 absorption line. The laser light source 18 is a tunable laser and laser light 18a output from the laser light source 18 is continuous light having a constant light quantity.

Laser light 18a supplied through the optical connector 20 propagates in the +X direction and is incident on a polarization plate 21. Laser light 18a passed through the polarization plate 21 becomes linearly polarized light. Laser light 18a is sequentially incident on a half mirror 22, a half mirror 23, a half mirror 24, and a reflection mirror 25. In a case where laser light 18a emitted from the laser light source 18 is linearly polarized light, the polarization plate 21 may not be provided.

The half mirrors 22, 23, and 24 reflect some of laser light fluxes 18a to be propagated in the +Y direction and allow some of laser light fluxes 18a to pass through to be propagated in the +X direction. The reflection mirror 25 reflects all of incident laser light fluxes 18a to the +Y direction. Laser light 18a is divided into light fluxes to be propagated along four light paths by the half mirrors 22, 23, and 24 and the reflection mirror 25. Reflectances of the half mirrors 22, 23, and 24 and the reflection mirror 25 are set so that light intensities of laser light 18a in respective light paths become equal.

Next, as illustrated in FIG. 2, laser light 18a is sequentially incident on a half mirror 26, a half mirror 27, a half mirror 28, and a reflection mirror 29. The half mirrors 26, 27, and 28 reflect some of laser light fluxes 18a to be propagated in the +Z direction and allow some of laser light fluxes 18a to pass through to be propagated in the +Y direction. The reflection mirror 29 reflects all of incident laser light fluxes 18a to the +Z direction.

Laser light 18a propagating in a single light path is divided into light fluxes to be propagated along four light paths by the half mirrors 26, 27, and 28 and the reflection mirror 29. Reflectances of the half mirrors 26, 27, and 28 and the reflection mirror 29 are set so that light intensities of laser light 18a in respective light paths become equal. Accordingly, laser light 18a is separated into light fluxes to be propagated along sixteen light paths. Reflectances of the half mirrors 22, 23, 24, 26, 27, and 28 and the reflection mirrors 25 and 29 are set so that light intensities of laser light 18a in respective light paths become equal.

The laser light source 18, the optical fiber 19, the optical connector 20, the polarization plate 21, and the half mirrors 22, 23, 24, 26, 27, and 28 and the reflection mirrors 25 and 29 constitute a light flux emitting unit 30 which emits laser light 18a as a plurality of light fluxes (sixteen light fluxes in the illustrated example) in the Z-axis direction.

Sixteen gas cells 12 of four rows and four columns are provided in each light path of laser light 18a in the +Z direction side of the half mirrors 26, 27, and 28 and the reflection mirror 29. Laser light fluxes 18a reflected by the half mirrors 26, 27, and 28 and the reflection mirror 29 pass through the gas cell 12. The gas cell 12 is a box having a void therein and the void is filled with alkali metal gas. Alkali metal is not particularly limited and, for example, potassium (K), rubidium (Rb), cesium (Cs), or the like is used. In the present embodiment, for example, cesium may be used in alkali metal.

A polarization separator 13 is installed in the +Z direction side of each gas cell 12. The polarization separator 13 is an element to separate incident laser light 18a into laser light fluxes 18a of two polarized components orthogonal to each other. In the polarization separator 13, for example, a Wollaston prism, a polarization flux splitter, or the like can be used.

A first detector 14 is installed at the +Z direction side of the polarization separator 13 and a second detector 15 is installed at the +Y direction side of the polarization separator 13. Laser light 18a passed through the polarization separator 13 is incident on the first detector 14 and laser light 18a reflected by the polarization separator 13 is incident on the second detector 15. The first detector 14 and the second detector 15 output currents according to light quantities of the incident laser light fluxes 18a to the processing device 2.

When the first detector 14 and the second detector 15 generate a magnetic field, there is a possibility that the magnetic field may influence on measurement and thus, the first detector 14 and the second detector 15 are preferably made of a non-magnetic material. The first magnetic sensor 10 includes heaters 16 installed at both sides in the X-axis direction and both sides in the Y-axis direction. The heater 16 preferably has a structure in which a magnetic field is not generated and it is possible to use, for example, a heater in which steam and hot air is allowed to pass through a flow passage to heat the gas cell. Instead of the heater, the gas cell 12 may be dielectrically heated by a high-frequency voltage.

The first magnetic sensor 10 is disposed in the +Z direction side of the subject 9 (see FIG. 1). A magnetic vector produced by the subject 9 enters the first magnetic sensor 10 from the −Z direction side. The magnetic vector passes through the half mirrors 26 to 28 and the reflection mirror 29, passes through the gas cell 12, and then, passes through the polarization separator 13, and is output from the first magnetic sensor 10.

Cesium within the gas cell 12 is heated to become a gas state. Cesium gas is irradiated with laser light 18a which is linearly polarized such that cesium atoms are excited and orientations of magnetic moments are aligned. In this state, when the magnetic vector passes through the gas cell 12, the magnetic moment of cesium atoms is subjected to precession by the magnetic field of the magnetic vector. The precession is called Larmor precession.

The magnitude of the Larmor precession has positive correlation with intensity of the magnetic field of the magnetic vector. The Larmor precession causes the polarization plane of laser light 18a to be rotated. There is positive correlation between the magnitude of the Larmor precession and a change amount of a rotation angle of the polarization plane of laser light 18a. Accordingly, there is positive correlation between the intensity of the magnetic field and the change amount of the rotation angle of the polarization plane of laser light 18a.

The polarization separator 13 separates laser light 18a into linearly polarized light beams of two components orthogonal to each other. The first detector 14 and the second detector 15 detect intensities of linearly polarized light beams of two components orthogonal to each other. With this, the first detector 14 and the second detector 15 can detect a rotation angle of a polarization plane of laser light 18a. The processing device 2 can compute the magnetic field from change of the rotation angle of the polarization plane of laser light 18a. The polarization separator 13, the first detector 14, and the second detector 15 constitute a light detection element 40 that detects optical characteristics of laser light 18a (light flux).

A detection unit 11 is constituted with the gas cell 12, the polarization separator 13, the first detector 14, and the second detector 15. The detection unit 11 is a sensor called optically pumped type magnetic sensor or optically pumped atomic magnetic sensor. Sensitivity of the detection unit 11 is high in the Z-axis direction, is low or becomes zero in the direction orthogonal to the Z-axis direction. As illustrated in FIG. 3, for example, sixteen detection units 11 of four rows and four columns are disposed in the first magnetic sensor 10. The number and disposition of the detection units 11 in the first magnetic sensor 10 are not particularly limited. The detection units 11 of three rows or less or five rows or more may be provided. Similarly, the detection units 11 of three columns or less or five columns or more may be provided. The more the number of the detection units 11, the higher the spatial resolution can be.

### 1. 3. Configuration of Second Magnetic Sensor

Although flowing of an external magnetic field into measurement target space in which the first magnetic sensor 10 is disposed is suppressed by the magnetic shield device 6 (see FIG. 1), it is difficult to completely prevent the external magnetic field from flowing into the measurement target space. The second magnetic sensor 100 is for measuring, for example, an environmental magnetic field (magnetic noise) in the measurement target space in which the first magnetic sensor 10 is disposed. The second magnetic sensor 100 may detect a measurement target magnetic field (heart magnetic field) together with the environmental magnetic field (magnetic noise).

The second magnetic sensor 100 has detection axes of the X-axis direction, the Y-axis direction, and the Z-axis direction. With this, it is possible to accurately estimate distribution of a planar environmental magnetic field or distribution of a spatial environmental magnetic field in a periphery of the second magnetic sensor 100 compared to, for example, a case where the second magnetic sensor 100 has only the detection axis of the Z-axis direction. Detailed configuration of the second magnetic sensor 100 will be described in the paragraph of “2. Magnetic sensor” to be described later.

### 1.4. Configuration of Processing Device

FIG. 4 is a diagram illustrating an example of a configuration of a processing device 2. As illustrated in FIG. 4, the processing device 2 is configured to include a manipulation unit 110, a display unit 112, a storing unit 114, and an operation unit 116.

The manipulation unit 110 is for inputting information (various instructions such as instruction to start measurement of the magnetic field or measurement condition) needed for processing to be performed by the operation unit 116 and may be, for example, various switches such as a button switch, a lever switch, or a dial switch, a touch panel, a keyboard, and a mouse.

The display unit 112 is for displaying a processing result of the operation unit 116 as characters, a graph, a table, animation, and other images, and may be, for example, a liquid crystal display (LCD) and an electroluminescence display (EL). Functions of the manipulation unit 110 and the display unit 112 may be implemented by a single touch panel type display.

The storing unit 114 is for storing a program or data used for performing various processing by the operation unit 116 and is configured with, for example, various integrated circuit (IC) memories such as a read only memory (ROM), a flash ROM, or a random access memory (RAM), a recording medium such as a hard disk or a memory card. The storing unit 114 is used as a working area of the operation unit 116 and temporarily stores, for example, an operation result executed according to various programs by the processing-operational unit 140. Furthermore, the storing unit 130 may store a piece of data that needs to be stored for a long time among pieces of data generated by processing of the processing-operational unit 140.

The operation unit 116 is implemented by, for example, a microprocessor such as a central processing unit (CPU) and performs correction processing or magnetic field computation processing described above. Specifically, the operation unit 116 acquires a first measurement value of the first magnetic sensor 10 and a second measurement value of the second magnetic sensor 100 and performs magnetic field computation processing based on the first measurement value and the second measurement value. With this, in the magnetic field measurement apparatus 1, it is possible to make the influence by the environmental magnetic field (magnetic noise) small in the measurement target space in which the first magnetic sensor 10 is disposed and more accurately measure a magnetic field such as the heart magnetic field or the brain magnetic field, which becomes a measurement target, of a living body.

### 2. Magnetic Sensor

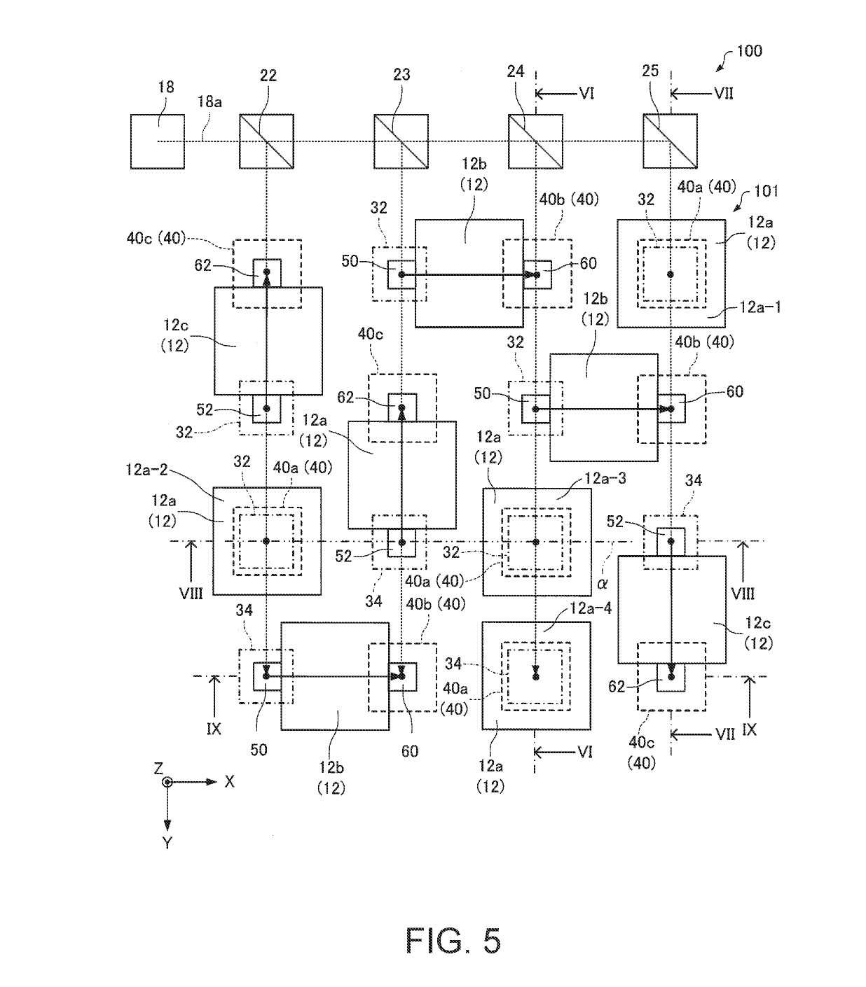

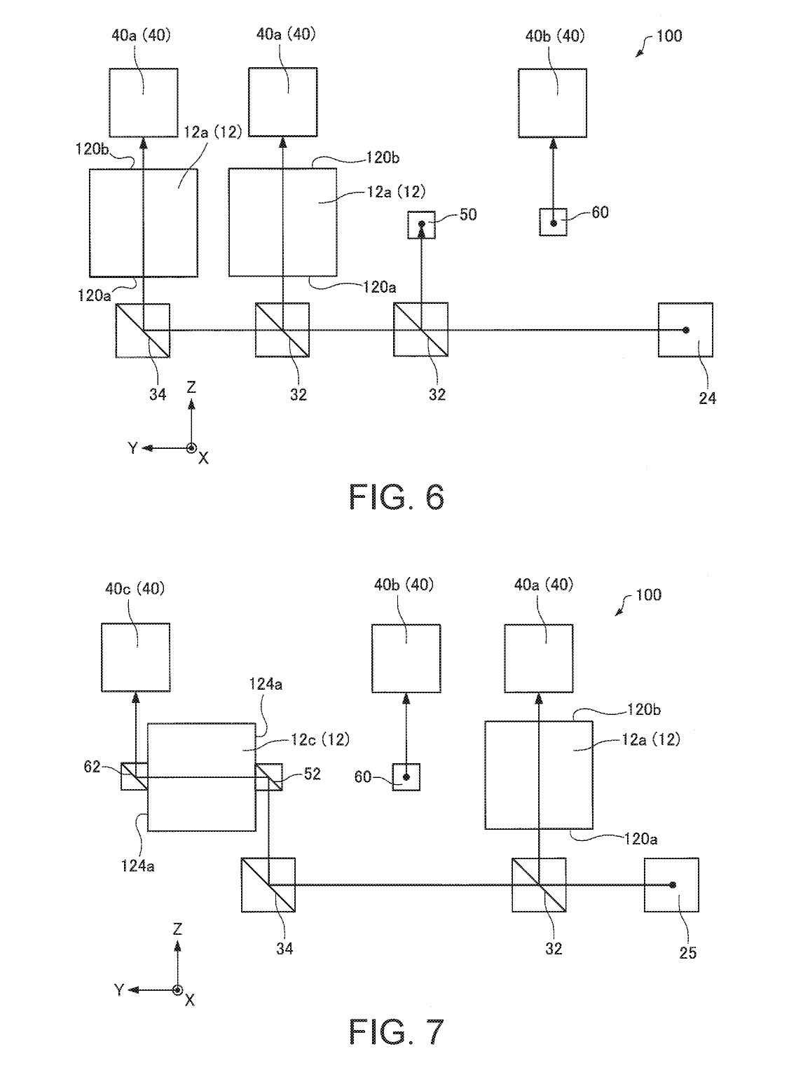

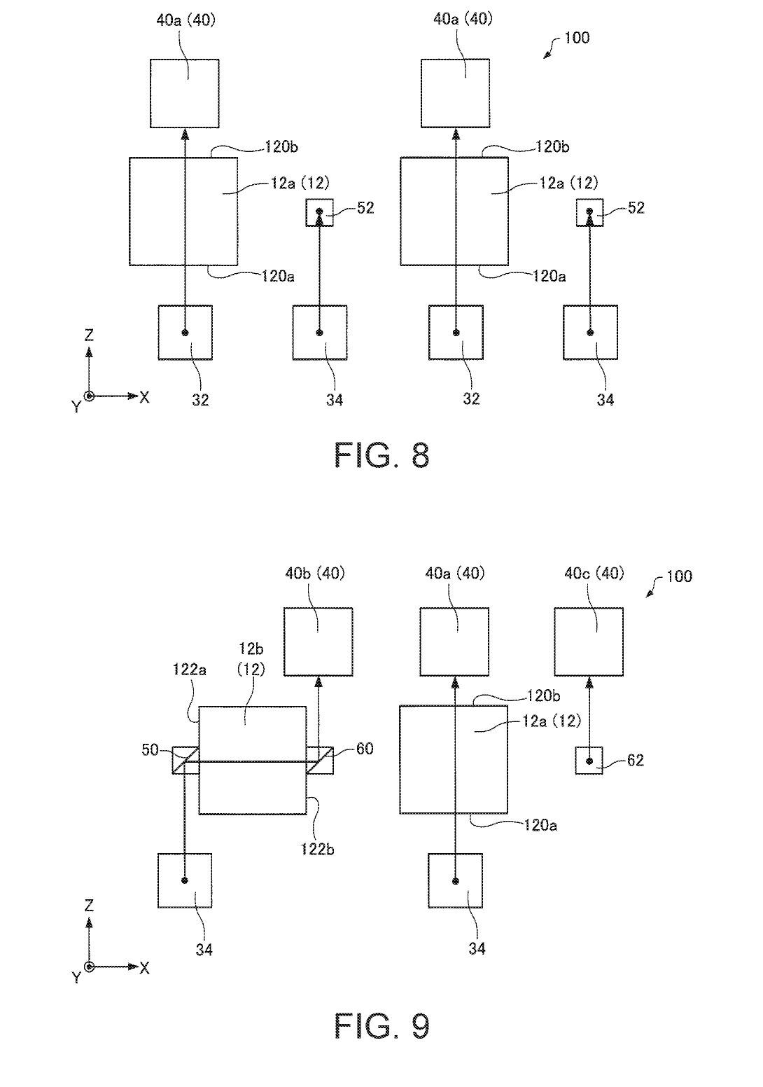

Next, the second magnetic sensor 100 according to the present embodiment (in the following, simply referred to as a “magnetic sensor 100”) will be described with reference to the accompanying drawings. FIG. 5 is a plan view schematically illustrating the magnetic sensor 100 according to the present embodiment. FIG. 6 to FIG. 9 are cross-sectional views schematically illustrating the magnetic sensor 100 according to the present embodiment. FIG. 6 is a cross-sectional view taken along VI-VI line of FIG. 5, FIG. 7 is a cross-sectional view taken along VII-VII line of FIG. 5, FIG. 8 is a cross-sectional view taken along VIII-VIII line of FIG. 5, and FIG. 9 is a cross-sectional view taken along IX-IX line of FIG. 5. In FIG. 5 to FIG. 9 and FIG. 10 to FIG. 14 illustrated in the following, the X-axis, the Y-axis, and the Z-axis are illustrated as three axes orthogonal to each other.

In the following, in the magnetic sensor 100 according to the present embodiment, constitutional members having the same function as those of the first magnetic sensor 10 according to the present embodiment described above are assigned the same reference numerals and detailed description thereof will be omitted. This is also similarly applied to a second magnetic sensor according to a modification example of the present embodiment which will be described in the following.

As illustrated in FIG. 5 to FIG. 9, the magnetic sensor 100 includes the gas cell 12, the light flux emitting unit 30, the light detection element 40, light flux benders 50 and 52, and light flux guide portions 60 and 62 . The magnetic sensor 100 may include the heater 16 and the substrate 17 (see FIG. 3). For convenience, in FIG. 5, the light detection element 40 is illustrated by a broken line.

The light flux emitting unit 30 emits a plurality of light fluxes in a first direction (Z-axis direction and direction parallel to Z-axis direction in the illustrated example). The light flux emitting unit 30 includes the laser light source 18, the half mirrors 22, 23, 24, and 32, and the reflection mirrors 25 and 34. The light flux emitting unit 30 may include the optical fiber 19, the optical connector 20, and the polarization plate 21 (see FIG. 3). For convenience, in FIG. 5, half mirror 32 and the reflection mirror 34 are illustrated by one-dot chain line.

As illustrated in FIG. 5, laser light 18a emitted from the laser light source 18 propagates in the +X direction and then, is sequentially incident on the half mirrors 22, 23, and 24 and the reflection mirror 25. The half mirrors 22, 23, and 24 reflect some of laser light beams 18a so as to be propagated in the +Y direction and allow some of laser light beams 18a to pass through so as to be propagated in the +X direction. The reflection mirror 25 reflects all of incident laser light beams 18a to the +Y direction.

As illustrated in FIG. 7, laser light 18a reflected from the reflection mirror 25 is sequentially incident on the half mirror 32 and the reflection mirror 34. The half mirror 32 reflects some of laser light beams 18a so as to be propagated in the +Z direction and allows some of laser light beams 18a to pass through so as to be propagated in the +Y direction. The reflection mirror 34 reflects all of incident laser light beams 18a to the +Z direction.

As illustrated in FIG. 6, laser light 18a reflected from the half mirror 24 is sequentially incident on two half mirrors 32 and the reflection mirror 34. As illustrated in FIG. 5, laser light 18a reflected from the half mirror 23 is sequentially incident on the half mirrors 32 and the reflection mirror 34. Laser light 18a reflected from the half mirror 22 is sequentially incident on two half mirrors 32 and the reflection mirror 34.

In the illustrated example, laser light 18a propagating along a single light path is separated into light beams to be propagated along ten light paths to become a plurality of light fluxes propagating in the +Z direction by the half mirrors 22, 23, 24, and 32 and the reflection mirrors 25 and 34. Six half mirrors 32 are provided and four reflection mirrors 34 are provided. The number of light paths of laser light 18a can be determined by the number of the half mirror 32 and the reflection mirror 34. The number of the half mirror 32 and the reflection mirror 34 is not particularly limited. For example, reflectances of the half mirrors 22, 23, 24, and 32 and the reflection mirrors 25 and 34 are set so that light intensities of laser light beams 18a in respective light paths become equal. A plurality of light fluxes propagating in the +Z direction may be disposed in an array form when viewed from the Z-axis direction. The plurality of light fluxes propagating in the +Z direction may be disposed at equal intervals when viewed from the Z-axis direction.

The first light flux bender 50 bends some of a plurality of light fluxes (separated laser light beams 18a) emitted from the light flux emitting unit 30 toward a second direction (the X-axis direction and the direction parallel to the X-axis direction in the illustrated example) different from the Z-axis direction. Specifically, the first light flux bender 50 is a reflection mirror and reflects laser light 18a propagating in the +Z direction to the X-axis direction. In the illustrated example, three first light flux benders 50 are provided to reflect three light fluxes among ten light fluxes to the X-axis direction. The first light flux bender 50 is provided in the +Z direction of the half mirror 32 or the reflection mirror 34.

The second light flux bender 52 bends some of a plurality of light fluxes (separated laser light beams 18a) emitted from the light flux emitting unit 30 toward a third direction (the Y-axis direction and the direction parallel to the Y-axis direction in the illustrated example) different from the X-axis direction and the Z-axis direction. Specifically, the second light flux bender 52 is a reflection mirror and reflects laser light 18a propagating in the +Z direction to the Y-axis direction. The first direction, the second direction, and the third direction are directions orthogonal to each other. In the illustrated example, three second light flux benders 52 are provided to reflect three light fluxes among ten light fluxes to the Y-axis direction. The second light flux bender 52 is provided in the +Z direction of the half mirror 32 or the reflection mirror 34. The light flux benders 50 and 52 may be attached to the gas cell 12 and may be attached to a substrate (not illustrated).

Although not illustrated, the first light flux bender 50 and the second light flux bender 52 may be elements such as an optical fiber or an optical waveguide made of a semiconductor as long as the elements can respectively bend the light fluxes toward the second direction and the third direction.

The gas cell 12 accommodates a medium which changes optical characteristics of the light flux depending on the magnitude of the magnetic field. Specifically, the gas cell 12 accommodates gaseous alkali metal (alkali metal vapor). Alkali metal absorbs light fluxes having oscillation wavelengths of laser light 18a and is optically pumped. In this state, alkali metal interacts with the applied magnetic field to thereby change a polarization plane of light transmitted through the gas cell 12 by effects of circular birefringence or a linear dichroic property depending on the magnitude of the magnetic field.

As illustrated in FIG. 5, for example, the gas cell 12 is provided between the light fluxes (separated laser light beams 18a) propagating in the Z-axis direction when viewed from the Z-axis direction. In FIG. 5, the light fluxes propagating in the Z-axis direction are illustrated by the black dots. In the illustrated example, the shape of the gas cell is a cube, but is not particularly limited. The material of the gas cell 12 is, for example, quartz glass and borosilicate glass.

A plurality of the gas cells 12 may be provided according to the number of the light fluxes emitted from the light flux emitting unit 30. The plurality of gas cells 12 may be supported on a substrate (not illustrated). In the illustrated example, ten gas cells 12 are provided. The plurality of gas cells 12 are classified into a first gas cell (first cell) 12a, a second gas cell (second cell) 12b, and a third gas cells (third cell) 12c. For example, the first gas cells 12a, the second gas cell 12b, and the third gas cell 12c are respectively provided by three or more.

The light flux emitted from the light flux emitting unit 30 and propagating in the Z-axis direction is incident on the first gas cells 12a. The first gas cell 12a is a gas cell through which the light flux passes in the Z-axis direction. The first gas cell 12a is provided in the +Z direction of the half mirror 32 or the reflection mirror 34. In the illustrated example, four first gas cells 12a are provided.

As illustrated in FIG. 6 to FIG. 9, the first gas cell 12a includes a light incidence surface 120a onto which the light flux is incident and a light emission surface 120b from which the light flux is emitted. The light incidence surface 120a and the light emission surface 120b are opposed to each other in the Z-axis direction. That is, the light incidence surface 120a and the light emission surface 120b include the normal line in the Z-axis direction (the normal line extending in the Z-axis direction).

The light flux bent toward the X-axis direction from the first light flux bender 50 is incident on the second gas cell 12b. The second gas cell 12b is a gas cell through which the light flux passes in the X-axis direction. In the illustrated example, three second gas cells 12b are provided.

The second gas cell 12b includes a light incidence surface (first surface) 122a onto which the light flux is incident and a light emission surface (second surface) 122b from which the light flux is emitted. The light incidence surface 122a and the light emission surface 122b are opposed to each other in the X-axis direction. That is, the light incidence surface 122a and the light emission surface 122b include the normal line in the X-axis direction (the normal line extending in the X-axis direction).

The light flux bent toward the Y-axis direction from the second light flux bender 52 is incident on the third gas cell 12c. The third gas cell 12c is a gas cell through which the light flux passes in the Y-axis direction. In the illustrated example, three third gas cells 12c are provided. For example, the number of the second gas cells 12b and the number of third gas cells 12c are the same.

The third gas cell 12c includes a light incidence surface 124a onto which the light flux is incident and a light emission surface 124b from which the light flux is emitted. The light incidence surface 124a and the light emission surface 124b are opposed to each other in the Y-axis direction. That is, the light incidence surface 124a and the light emission surface 124b include the normal line in the Y-axis direction (the normal line extending in the X-axis direction).

The first gas cell 12a, the second gas cell 12b, and the third gas cell 12c are provided on the same plane. That is, the gas cells 12a, 12b, and 12c are provided so as to allow a predetermined plane to pass through. In the illustrated example, the gas cell 12a, 12b, and 12c are provided in the XY-plane (the side passing through the X-axis direction and the Y-axis direction).

Any of the first gas cell 12a, the second gas cell 12b, and the third gas cell 12c is provided by three or more and all of the centers of the cells provided by three or more are not aligned on a line (straight line). In the illustrated example, the gas cells 12a, 12b, and 12c are respectively provided by three or more. The centers of three or more first gas cells 12a are not aligned on a straight line. For example, as illustrated in FIG. 5, the centers of first gas cells 12a-1 and 12a-4 are not positioned on a virtual straight line a passing the centers of first gas cells 12a-2 and 12a-3. That is, the centers of first gas cells 12a-1 and 12a-4 are positioned to be separated from the virtual straight line α. Similarly, the centers of three or more second gas cells 12b are not aligned on a straight line. The centers of three or more third gas cells 12c are not aligned on a straight line. The light fluxes passed through the gas cells 12a, 12b, and 12c respectively pass through, for example, the centers of the gas cells 12a, 12b, and 12c.

The plurality of first gas cells 12a are preferably provided dispersively, the plurality of second gas cells 12b are preferably provided dispersively, and the plurality of third gas cells 12c are preferably provided dispersively. With this, the magnetic sensor 100 can accurately detect components in respective direction of the magnetic field.

The first light flux guide portion 60 guides the light flux emitted from the second gas cell 12b to a second light detection element 40b. Specifically, the first light flux guide portion 60 is a reflection mirror and reflects the light flux emitted from the second gas cell 12b and propagating in the X-axis direction to the +Z-axis direction. In the illustrated example, three first light flux guide portions 60 are provided in correspondence with the second gas cells 12b. Each second gas cell 12b is provided to be sandwiched between the first light flux bender 50 and the first light flux guide portion 60 in the X-axis direction. The first light flux bender 50 is, for example, an inclined mirror (first reflection mirror) provided at the light incidence surface 122a side of the second gas cell 12b and inclined at 45 degrees to the light incidence surface 122a. The first light flux guide portion 60 is, for example, an inclined mirror (second reflection mirror) provided at the light emission surface 122b side of the second gas cell 12b and inclined at 45 degrees to the light emission surface 122b.

The second light flux guide portion 62 guides the light flux emitted from the third gas cell 12c to a third light detection element 40c. Specifically, the second light flux guide portion 62 is a reflection mirror and reflects the light flux emitted from the third gas cell 12c and propagating in the Y-axis direction to the +Z direction. In the illustrated example, three second light flux guide portions 62 are provided in correspondence with the third gas cells 12c. Each third gas cell 12c is provided to be sandwiched between the second light flux bender 52 and the second light flux guide portion 62 in the Y-axis direction. The second light flux bender 52 is, for example, an inclined mirror provided at the light incidence surface 124a side of the third gas cell 12c and inclined at 45 degrees to the light incidence surface 124a. The second light flux guide portion 62 is, for example, an inclined mirror provided at the light emission surface 124b side of the third gas cell 12c and inclined at 45 degrees to the light emission surface 124b.

The first light flux guide portion 60 may be a phase compensation mirror reflecting the light flux emitted from the second gas cell 12b. The second light flux guide portion 62 may be a phase compensation mirror reflecting the light flux emitted from the third gas cell 12c. The light flux guide portions 60 and 62 reflect the light flux (incident light flux) while maintaining a phase difference between P wave and S wave of the light flux of which a polarization plane is rotated as it is. A portion of the light flux guide portions 60 and 62 onto which the light flux is incident may be configured with a dielectric multilayer film reflecting the light flux while maintaining a phase difference between P wave and S wave of the incident light flux as it is. The light flux guide portions 60 and 62 maybe attached to the gas cell 12 and may be attached to a substrate (not illustrated).

The light detection element 40 is configured to include the polarization separator 13, the first detector 14, and the second detector 15 (see FIG. 2). For convenience, the light detection element 40 is illustrated by being simplified in FIG. 5 to FIG. 9. The light detection elements 40 are classified into a first light detection element 40a, a second light detection element 40b, and a third light detection element 40c.

The light flux emitted from the first gas cell 12a is incident on the first light detection element 40a. The first light detection element 40a is provided in the +Z direction of the first gas cell 12a. The light flux emitted from the second gas cell 12b is incident on the second light detection element 40b through the first light flux guide portion 60. The second light detection element 40b is provided in the +Z direction of the first light flux guide portion 60. The light flux emitted from the third gas cell 12c is incident on the third light detection element 40c through the second light flux guide portion 62. The third light detection element 40c is provided in the +Z direction of the second light flux guide portion 62.

The light detection elements 40a, 40b, and 40c respectively detect optical characteristics of the light fluxes emitted from the gas cells 12a, 12b, and 12c. Specifically, the light detection elements 40a, 40b, and 40c respectively detect the rotation angles of the polarization planes of the light fluxes emitted from the gas cells 12a, 12b, and 12c. In a case where absolute intensity of the applied magnetic field is minute, the rotation angle of the polarization plane is proportional to the magnitude of the magnetic field component projected in a propagation direction of the light flux within the gas cell 12. Accordingly, a plurality of three kinds of the gas cells 12a, 12b and 12c, through which the light fluxes propagating in the Z-axis direction, the X-axis direction, and the Y-axis direction respectively pass, are provided to make it possible to detect magnetic field distribution of three-dimensional components in the magnetic sensor 100.

The gas cells 12a and 12b, the light detection elements 40a and 40b, the first light flux bender 50, and the first light flux guide portion 60 constitute a cell unit 101. The cell unit 101 may be configured to include the third gas cell 12c, the third light detection element 40c, the second light flux bender 52, and the second light flux guide portion 62.

The magnetic sensor 100 has, for example, the following features.

The magnetic sensor 100 includes the first gas cell 12a onto which the light flux emitted from the light flux emitting unit 30 and propagating in the first direction is incident and the second gas cell 12b onto which the light flux bent toward the second direction from the first light flux bender 50 is incident. For that reason, in the magnetic sensor 100, it is possible to detect components in a plurality of directions of the magnetic field without providing a plurality of pairs of Helmholtz coils in order to detect, for example, the components in the plurality of directions of the magnetic field. Accordingly, in the magnetic sensor 100, it is possible to detect components in the plurality of directions of the magnetic field and accurately detect the magnetic field with a simple configuration. Furthermore, in the magnetic sensor 100, a circuit for driving the pair of Helmholtz coils may not be provided in order to detect the components in the plurality of directions of the magnetic field.

The magnetic sensor 100 includes the third gas cell 12c onto which the light flux bent toward the third direction from the second light flux bender 52 is incident. Accordingly, in the magnetic sensor 100, it is possible to detect the components in three directions of the magnetic field.

In the magnetic sensor 100, the first direction, the second direction, and the third direction are orthogonal to each other. Accordingly, in the magnetic sensor 100, it is possible to detect components in three axial directions orthogonal to each other of the magnetic field.

In the magnetic sensor 100, any of the first gas cell 12a, the second gas cell 12b, and the third gas cell 12c is provided by three or more and all of the centers of the cells provided by three or more are not aligned on a straight line.

Here, although it is ideal that respective components Bₓ, B_{y}, and B_{z} of the magnetic field on calculation in certain time at an arbitrary point (x,y,z) match order of magnetic field distribution to be measured, it is assumed that the respective components Bₓ, B_{y}, and B_{z} are represented by first-order expressions of the following expressions (1), (2), and (3). In the following expressions (1), (2), and (3), a_{1X} to a_{4X}, a_{1Y} to a_{4Y}, and a_{1Z} to a_{4Z} are coefficients.

Bₓ=a₁ₓ+a₂ₓx+a₃ₓy+a₄ₓz (1)

B_{y}=a_{1y}+a_{2y}x+a_{3y}y+a_{4y}z (2)

B_{z}=a_{1z}+a_{2z}x+a_{3z}y+a_{4z}z (3)

Here, unknowns of the expressions (1) to (3) are twelve unknowns of the coefficients a₁ₓ to a₄ₓ, a_{1y} to a_{4y}, and a_{1z} to a_{4z}, but it is possible to create four relation expressions from the condition that both divergence and rotation of magnetic field which are nature of a magnetic field are zero and reduce the number of unknowns of equations (1) to (3) to eight unknowns. In other words, when eight cells are present, it is possible to obtain all coefficients. That is, although the number of two kinds of gas cells may be three and the number of one kind of gas cells may be two among the first to third gas cells, when the gas cells constituting any of the first to third gas cells are aligned on a straight line, an indefinite coefficient occurs and thus, it is not preferable. Accordingly, in the magnetic sensor 100, any of the first gas cell 12a, the second gas cell 12b, and the third gas cell 12c is provided by three or more and all of the centers of the cells provided by three or more are not aligned on a straight line and as a result, it is possible to more surely detect components in three directions, which are orthogonal to each other, of the magnetic field.

In the magnetic sensor 100, the number of the second gas cells 12b and the number of the third gas cells 12c are the same. For that reason, in the magnetic sensor 100, it is possible to detect the X-axis direction component and the Y-axis direction component of the magnetic field with the same accuracy. The magnetic sensor 100 is positioned, for example, in the Z-axis direction of the subject 9 and thus, the X-axis direction component and the Y-axis direction component of the magnetic field are preferably detected with the same accuracy.

In the magnetic sensor 100, the first gas cell 12a, the second gas cell 12b, and the third gas cell 12c are provided on the same plane. For that reason, in the magnetic sensor 100, it is possible to easily support the first gas cell 12a, the second gas cell 12b, and the third gas cell 12c by, for example a single substrate.

The magnetic sensor 100 includes the first light flux guide portion 60 guiding the light flux emitted from the second gas cell 12b to the second light detection element 40b. For that reason, in the magnetic sensor 100, it is possible to make the light flux emitted from the second gas cell 12b incident on the first light flux guide portion 60 by the first light flux guide portion 60.

In the magnetic sensor 100, the first light flux guide portion 60 is a phase compensation mirror reflecting the light flux emitted from the second gas cell 12b and the first light flux guide portion 60 reflects the light flux while maintaining a phase difference between P wave and S wave of the light flux of which a polarization plane is rotated as it is. For that reason, in the magnetic sensor 100, it is possible to suppress decrease in sensitivity of the second light detection element 40b. For example, when the light flux is reflected from the first light flux guide portion 60, if the phase difference between P wave and S wave in the light flux before reflection is different from that in the light flux after reflection, sensitivity of the second light detection element 40b may be decreased. In the magnetic sensor 100, it is possible to avoid such a problem.

In the magnetic sensor 100, the light flux emitting unit 30 emits a plurality of light fluxes in the first direction. For that reason, in the magnetic sensor 100, it is possible to make the light flux propagating in the first direction incident on the first gas cell 12a without using the light flux bender.

In the magnetic sensor 100, gaseous alkali metal is accommodated in the gas cells 12a, 12b, and 12c. For that reason, in the magnetic sensor 100, alkali metal interacts with the applied magnetic field to make it possible to change the polarization plane of light transmitted through the gas cells 12a, 12b, and 12c depending on the magnitude of the magnetic field.

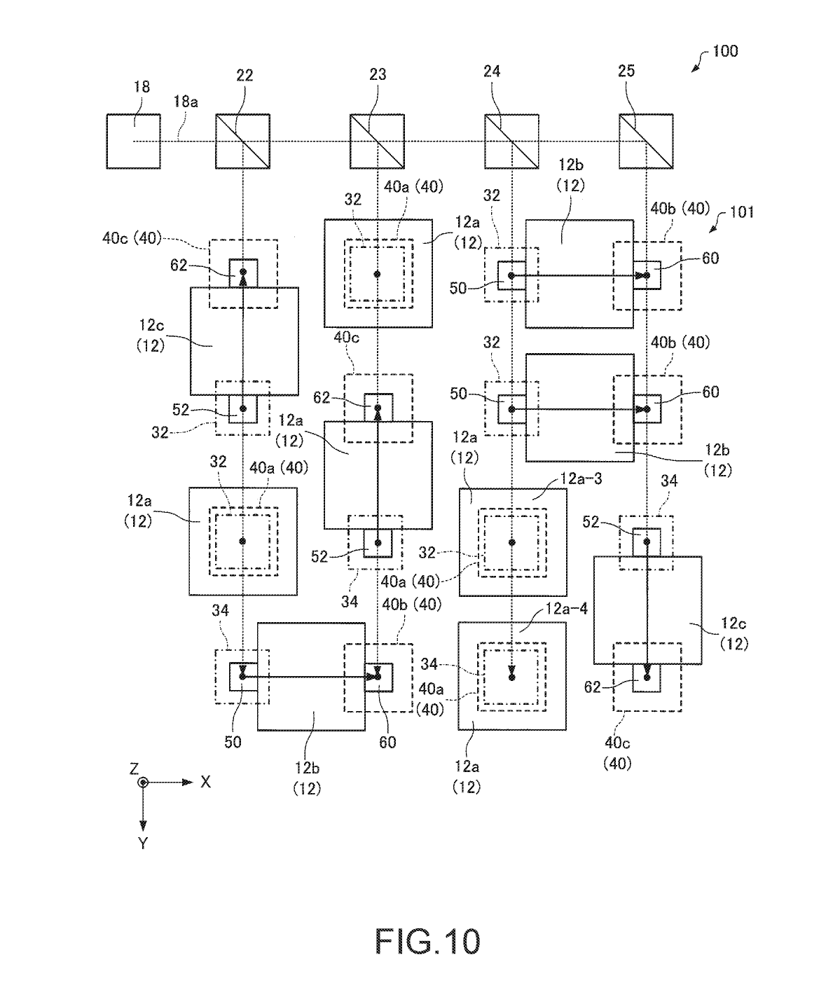

Disposition of the gas cells 12a, 12b, and 12c in the magnetic sensor 100 is not limited to the example of FIG. 5, but the gas cells 12a, 12b, and 12c may be disposed as in, for example, FIG. 10.

In the magnetic sensor 100, the directions in which light fluxes are passed through the gas cells 12a, 12b, and 12c are orthogonal to each other, but may not be orthogonal to each other as long as the directions are not parallel to each other. One of the second gas cell 12b and the third gas cell 12c may not be provided.

In the magnetic field measurement apparatus according to the invention, the plurality of magnetic sensors 100 may be aligned in the direction orthogonal to the Z-axis direction.

### 3. Modification Example of Magnetic Sensor

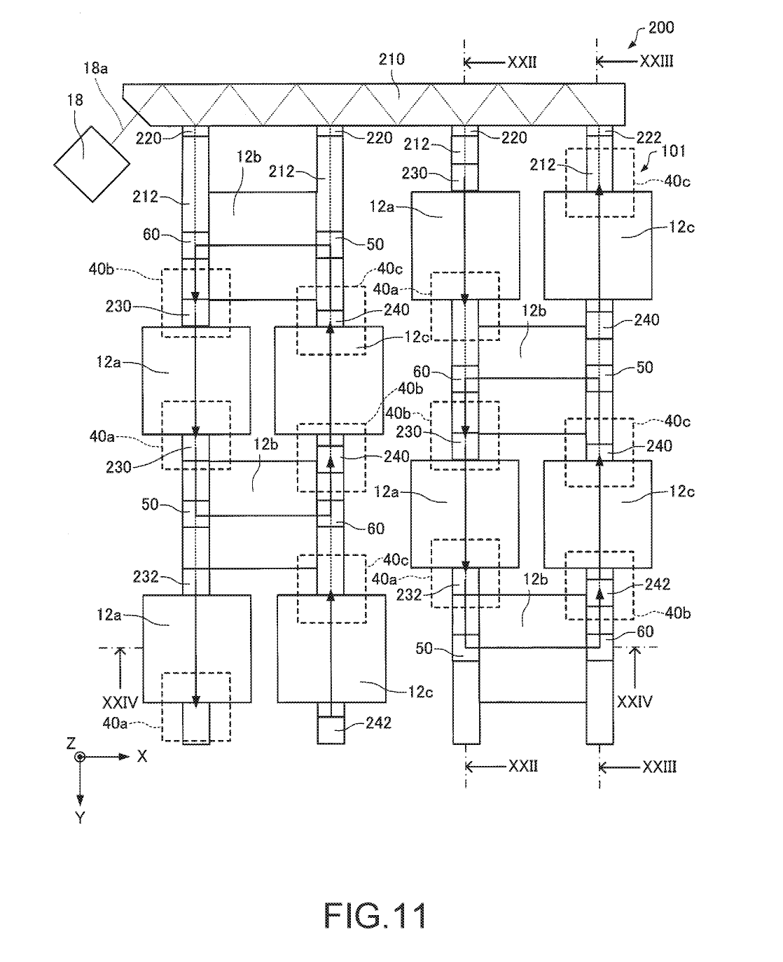

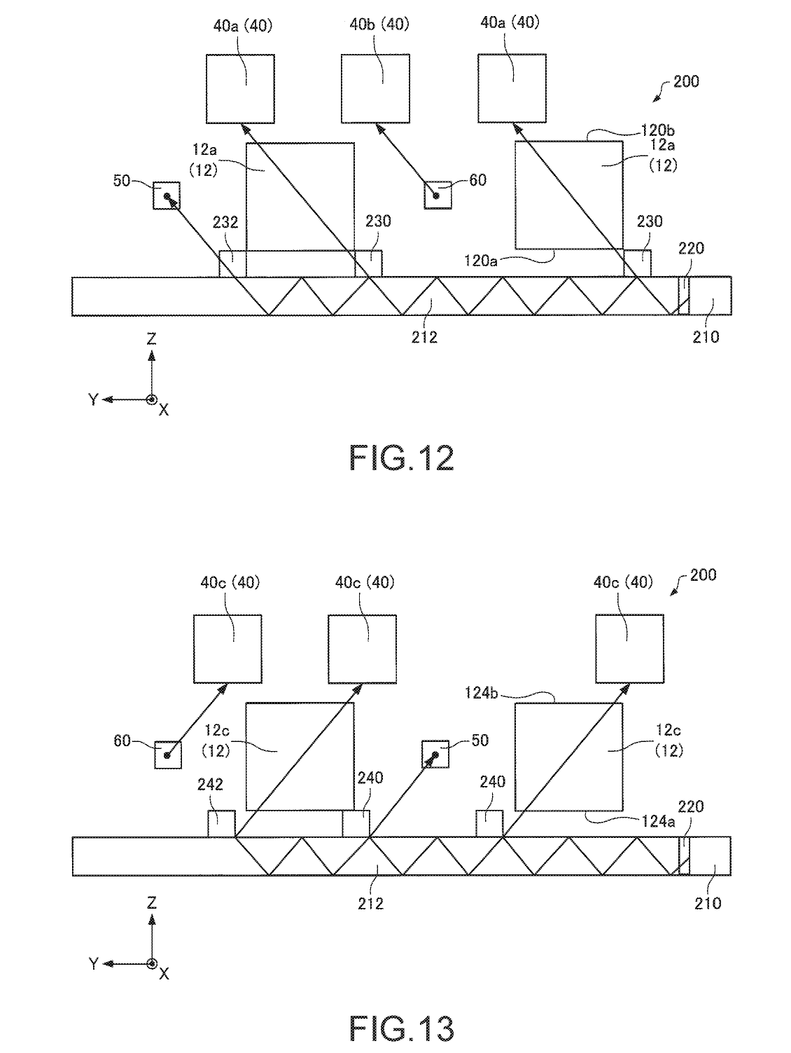

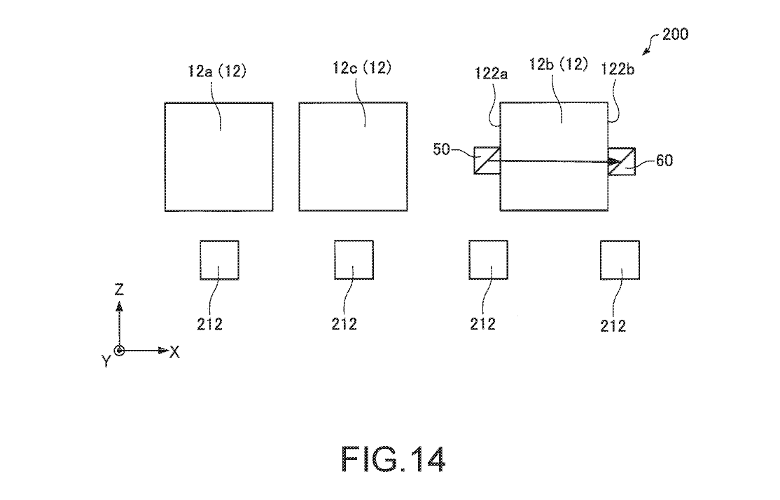

Next, a second magnetic sensor according to a modification example of the present embodiment will be described with reference to the drawings. FIG. 11 is a plan view schematically illustrating a second magnetic sensor 200 (in the following, simply referred to as a “magnetic sensor 200”) according to the modification example of the present embodiment. FIG. 12 to FIG. 14 are cross-sectional views schematically illustrating the magnetic sensor 200 according to the modification example of the present embodiment. FIG. 12 is a cross-sectional view taken along XXII-XXII line of FIG. 11, FIG. 13 is a cross-sectional view taken along XXIII-XXIII line of FIG. 11, and FIG. 14 is a cross-sectional view taken along XXIV-XXIV line of FIG. 11. In FIG. 11, light detection elements 40a, 40b, and 40c are illustrated by a broken line.

In the following, in the magnetic sensor 200 according to the modification example of the present embodiment, constitutional members having the same function as those of the magnetic sensor 100 according to the present embodiment described above are assigned the same reference numerals and detailed description thereof will be omitted.

In the magnetic sensor 100 described above, as illustrated in FIG. 5 to FIG. 9, the first gas cell 12a is a gas cell through which the light flux passes in the Z-axis direction, the second gas cell 12b is a gas cell through which the light flux passes in the X-axis direction, and the third gas cell 12c is a gas cell through which the light flux passes in the Y-axis direction.

In contrast, in the magnetic sensor 200, as illustrated in FIG. 11 to FIG. 14, the first gas cell 12a is a gas cell through which the light flux passes in a direction (for example, a direction inclined at 45 degrees with respect to the +Y direction from the Z-axis, first direction) inclined to the Y-axis and the Z-axis that are orthogonal to the X-axis direction, the second gas cell 12b is a gas cell through which the light flux passes in the X-axis direction (second direction), and the third gas cell 12c is a gas cell through which the light flux passes in a direction (for example, a direction inclined at 45 degrees with respect to the −Y direction from the Z-axis, third direction) inclined to the Y-axis and the Z-axis that are orthogonal to the X-axis direction.

The light flux emitting unit 30 of the magnetic sensor 200 includes a first light guide 210, a second light guide 212, and diffraction elements 220, 222, 230, 232, 240, and 242.

Laser light 18a emitted from the laser light source 18 is incident on the first light guide 210. In the illustrated example, the first light guide 210 is extended in the X-axis direction. Laser light 18a incident on the first light guide 210 propagates in the +X direction while being multi-reflected on an internal surface of the first light guide 210. The material of the first light guide 210 is, for example, glass and resin such as acrylic resin.

The diffraction elements 220 and 222 are provided in the first light guide 210. In the illustrated example, three diffraction elements 220 are provided and a single diffraction element 222 is provided. The diffraction element 222 takes out some of laser light fluxes 18a propagating in the first light guide 210 by diffraction and causes the laser light fluxes to propagate toward the +Y direction side. The diffraction element 222 is provided closer to the +X direction side than the diffraction element 220. The diffraction element 222 takes out all of incident laser light fluxes 18a by diffraction and causes the laser light fluxes to propagate toward the +Y direction side.

The second light guide 212 is connected to the first light guide 210. In the illustrated example, four second light guides 212 are provided. Laser light fluxes 18a taken out by the diffraction elements 220 and 222 are incident on the second light guide 212. The second light guide 212 is extended in the Y-axis direction. Laser light 18a incident on the second light guide 212 propagates in the +Y direction while being multi-reflected on an internal surface of the second light guide 212. The material of the second light guide 212 is the same as, for example, that of the first light guide 210.

The diffraction elements 230 and 232 are provided in the second light guide 212. In the illustrated example, four diffraction elements 230 are provided and two diffraction elements 232 are provided. The diffraction element 232 is provided closer to the +Y direction side than the diffraction element 230. The diffraction element 230 takes out some of laser light fluxes 18a propagating in the second light guide 212 by diffraction and causes the laser light fluxes to propagate toward the first direction. The diffraction element 232 takes out all of incident laser light fluxes 18a by diffraction and causes the laser light fluxes to propagate toward the first direction. Laser light 18a propagating in the first direction is incident on the first gas cell 12a or the first light flux bender 50.

The diffraction elements 240 and 242 are provided in the second light guide 212. In the illustrated example, four diffraction elements 240 are provided and two diffraction elements 242 are provided. The diffraction element 242 is provided closer to the +Y direction side than the diffraction element 240. The diffraction element 240 takes out some of laser light fluxes 18a propagating in the second light guide 212 by diffraction and causes the laser light fluxes to propagate toward the third direction. The diffraction element 242 takes out all of incident laser light fluxes 18a by diffraction and causes the laser light fluxes to propagate toward the third direction. Laser light 18a propagating in the third direction is incident on the third gas cell 12c or the first light flux bender 50.

In the illustrated example, laser light 18a propagating along a single light path is separated into light fluxes to be propagated along twelve light paths by the diffraction elements 220, 222, 230, 232, 240, and 242. For example, the diffraction elements 220, 222, 230, 232, 240, and 242 are designed so that light intensities of laser light 18a in respective light paths become equal. The diffraction elements 220, 222, 230, 232, 240, and 242 may be formed on a transparent substrate (not illustrated) by a nano-imprint method or the like. In the illustrated example, each of the gas cells 12a, 12b, and 12c is provided by four.

In the magnetic sensor 200, it is possible to divide laser light 18a into laser light fluxes of three directions even without providing the second light flux bender 52 or the second light flux guide portion 62 as in the magnetic sensor 100.

The invention includes a configuration (for example, a configuration having the same function, method, and effect or a configuration having the same object and effect) which is substantially the same as the configuration described in the embodiment. The invention includes a configuration in which a non-essential portion of the configuration described in the embodiment is replaced with another constitutional element. The invention includes a configuration by which the same effect as that of the configuration described in the embodiment can be obtained or a configuration by which the same object can be achieved. The invention includes a configuration obtained by adding a known technique to the configuration described in the embodiment.

The entire disclosure of Japanese Patent Application No. 2016-216089 filed Nov. 4, 2016 is expressly incorporated by reference herein.

What is claimed is:

1. A magnetic sensor comprising:
a light flux emitting unit that emits a plurality of light fluxes;
a first cell onto which a light flux, which is emitted from the light flux emitting unit and which propagates in a first direction, is incident and that accommodates a medium which changes optical characteristics of the light flux depending on a magnitude of a magnetic field;
a first light flux bender that bends some of the plurality of light fluxes emitted from the light flux emitting unit in a second direction different from the first direction;
a second cell onto which the light flux, which is bent in the second direction in the first light flux bender, is incident and that accommodates a medium which changes optical characteristics of the light flux depending on the magnitude of the magnetic field;
a first light detection element that detects optical characteristics of a light flux emitted from the first cell; and
a second light detection element that detects optical characteristics of a light flux emitted from the second cell.

2. The magnetic sensor according to claim 1, further comprising:
a second light flux bender that bends some of the plurality of light fluxes emitted from the light flux emitting unit in a third direction different from the first direction and the second direction;
a third cell onto which the light flux, which is bent in the third direction in the second light flux bender, is incident and that accommodates a medium which changes optical characteristics of the light flux depending on the magnitude of the magnetic field; and
a third light detection element that detects optical characteristics of the light flux emitted from the third cell.

3. The magnetic sensor according to claim 2,
wherein the first direction, the second direction, and the third direction are orthogonal to each other.

4. The magnetic sensor according to claim 2,
wherein any of the first cell, the second cell, and the third cell is provided with a quantity of three or more, and
all of the centers of the cells provided with a quantity of three or more are not aligned on a straight line.

5. The magnetic sensor according to claim 2, wherein the number of the second cells and the number of the third cells are the same.

6. The magnetic sensor according to claim 2,
wherein the first cells, the second cells, and the third cells are provided on the same plane.

7. The magnetic sensor according to claim 1, further comprising:
a light flux guide portion that guides the light flux emitted from the second cell to the second light detection element.

8. The magnetic sensor according to claim 7,
wherein the light flux guide portion is a phase compensation mirror reflecting the light flux emitted from the second cell, and
the light flux guide portion reflects a light flux while maintaining a phase difference between P wave and S wave of the light flux of which a polarization plane is rotated as it is.

9. The magnetic sensor according to claim 1,
wherein the light flux emitting unit emits the plurality of light fluxes in the first direction.

10. The magnetic sensor according to claim 1,
wherein the medium is gaseous alkali metal.

11. A cell unit comprising:
a first cell that accommodates a medium changing optical characteristics of a light flux depending on a magnitude of a magnetic field;
a first light detection element that is provided in a first direction of the first cell and detects optical characteristics of the light flux;
a second cell that accommodates a medium changing optical characteristics of the light flux depending on the magnitude of the magnetic field and has a first surface and a second surface opposing to each other in a second direction orthogonal to the first direction;
a first reflection mirror that is provided in the first surface side and inclined at 45 degrees to the first surface;
a second reflection mirror that is provided in the second surface side and inclined at 45 degrees to the second surface; and
a second light detection element that is provided in a first direction of the second reflection mirror and detects optical characteristics of the light flux.
